(19) United States
(12) Patent Application Publication
(10) Pub. No.: US 2025/0025351 A1
(43) Pub. Date: Jan. 23, 2025

(54) Menstrual Fluid Absorbent Device

(71) Applicants: Sherrie Paymon, Pontiac, MI (US); Kellie Ragland, Detroit, MI (US)

(72) Inventors: Sherrie Paymon, Pontiac, MI (US); Kellie Ragland, Detroit, MI (US)

(21) Appl. No.: 18/676,008

(22) Filed: May 28, 2024

Related U.S. Application Data
(60) Provisional application No. 63/514,147, filed on Jul. 18, 2023.

(51) Int. Cl. A61F 13/47

(52) U.S. Cl. CPC A61F 13/4704

(57) ABSTRACT

A menstrual flow absorbent disposable device designed for feminine hygiene is disclosed. The device is generally square or rectilinear, with dimensions ranging from 2.5″×2.5″ to 3″×3.5″ and is bent into a V-shape for optimal fit and absorption. In some embodiments, the device is pre-bent into the V-shape. The compact size and design of the device does not interfere with the use of a sanitary pad, enhancing menstrual fluid management without impacting the stability or efficacy of the pad. The device is made of superabsorbent biodegradable polymers, flushable cellulose fibers, and flushable nonwoven fabrics, is environmentally friendly and skin friendly. The device is specifically shaped to fit comfortably and discreetly between the buttocks or gluteus maximus muscles, easily conforming to the contours of the buttock or intergluteal cleft.

### CROSS-REFERENCE TO RELATED APPLICATION

The present application claims priority to, and the benefit of, U.S. Provisional Application No. 63/514,147, which was filed on Jul. 18, 2023 and is incorporated herein by reference in its entirety.

### FIELD OF THE INVENTION

The present invention generally relates to the field of menstrual fluid absorbent devices. More specifically, the present invention relates to a novel menstrual device that allows women to walk, jog, exercise, and/or sleep on their back without worry. The device is a compact V-shaped absorbent article that is placed between the buttocks (i.e., in the intergluteal cleft) to catch any leaking blood while a user using the device is lying on the back. The device functions as an additional barrier between bodily fluids and clothing. The device is flushable and can be flushed in a toilet for easy disposal. Accordingly, the present disclosure makes specific reference thereto. Nonetheless, it is to be appreciated that aspects of the present invention are also equally applicable to other like applications, devices, and methods of manufacture.

### BACKGROUND

By way of background, menstruation is a natural part of the female reproductive cycle. Mensuration serves as a monthly indicator of a woman's fertility and reproductive health. The menstrual cycle is orchestrated by hormonal changes that prepare the body for the possibility of pregnancy. During the menstrual cycle, the lining of the uterus gradually thickens and prepares for the possibility of pregnancy and if pregnancy does not occur, the body sheds the lining through the vagina, resulting in menstruation. The shedding of the uterine lining, along with blood and other fluids, causes menstrual flow. The average menstrual cycle lasts around 28 days, although the cycle can range from 21 to 35 days.

The amount of menstrual flow varies significantly from person to person. Some women experience a light flow, where bleeding is minimal and can be managed with lighter absorbent products, while others have a heavy flow, necessitating more substantial protection. Heavy flow can lead to leaks even with the use of traditional menstruation products like sanitary pads, tampons, or menstrual cups, which can be embarrassing and inconvenient. The leaks not only cause inconvenience and discomfort but also lead to clothing and bedding stains. Stains can impact not only clothing but also bed sheets and mattresses when a person is lying down or sleeping. Women have to frequently change their mensuration absorption products which is frustrating and time consuming. Therefore, females desire a more effective menstruation tool for absorbing excess body fluids and preventing leaks during a menstrual cycle.

Therefore, there exists a long felt need in the art for an improved mensuration tool for absorbing excess body fluids to prevent leaks. Additionally, there is a long felt need in the art for an improved mensuration tool that can be used in place of or along with conventional mensuration tools and products such as sanitary pads and tampons. Moreover, there is a long felt need in the art for a menstrual device that can be used by women during all activities to absorb menstrual flow. Further, there is a long felt need in the art for a menstrual device that is V-shaped and made of absorbent material to absorb any leaking menstrual flow. Furthermore, there is a long felt need in the art for a menstrual device that fits between the buttocks and within the intergluteal cleft of a female to catch leaking menstrual flow. Also, there is a long felt need in the art for a novel menstrual tool that provides absorption for extended time periods. Finally, there is a long felt need in the art for a menstruation tool that is flexible, that can be bent into a V-shape, and fits between the vagina and buttocks to absorb menstrual flow.

The subject matter disclosed and claimed herein, in one embodiment thereof, comprises a menstrual fluid absorbent device. The device is flushable and flexible and includes a generally square or rectilinear planar body with dimensions ranging from about 2.5 inches in length by 2.5 inches in width to about 3.0 inches in length by 3.5 inches in width. The device is adapted to be bent into a V-shape, facilitated by folding the body along the center thereof using a finger. The V-shape of the device conforms to the contour of the buttocks and the intergluteal cleft of a user for secure placement of the device between the gluteus maximus muscles. The device is made of one of superabsorbent biodegradable polymers, flushable cellulose fibers, and flushable nonwoven fabrics and disintegrates in water. After use, the device is removed from the buttocks or intergluteal cleft and is flushed in an environmentally friendly manner.

In this manner, the menstrual fluid absorbent device accomplishes all of the forgoing objectives and provides users with a menstruation tool capable of absorbing excess menstrual fluid to prevent leaks. The device is placed between the buttocks or gluteus maximus muscles to catch any leaking blood while the user using the device is lying on the back. The device can be used with a sanitary pad or other similar tools and functions as an additional barrier between bodily fluids and clothing. The absorbent device combines ergonomic design with specialized materials to provide a discreet, comfortable, and effective menstrual hygiene solution. The device can be used during various activities, including sleep, without the worry of leaks, while also being flushable for convenient disposal.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a menstrual flow absorbent flushable device. The device includes a planar body made of a fabric or similar material, having a generally square or rectilinear shape with dimensions ranging from about 2.5 inches in length by 2.5 inches in width, to about 3.0 inches in length by 3.5 inches in width. The device is adapted to be bent into a V-shape, facilitated by folding the body along the center (i.e., center line) thereof, thus conforming to the contours of the buttocks between the bum cheeks or gluteus maximus muscles of a user (i.e., intergluteal cleft) for secure placement of the device. The device is made of one of superabsorbent biodegradable polymers, flushable cellulose fibers, and flushable nonwoven fabrics and disintegrates in water.

In a further embodiment, a menstrual flow absorbent disposable device designed for use in conjunction with a sanitary pad is disclosed. The device has a V-shape flexible body and is adapted to be placed between the buttocks (i.e., within the intergluteal cleft) of a user for absorbing menstrual fluid and preventing staining of clothing. The device is placed between the buttocks or gluteus maximus muscles with the common edge of the V-shape body inserted in the space or intergluteal cleft between the buttocks, wherein the device is covered by a sanitary pad. After use, the device is pulled out of the buttocks or intergluteal cleft and is flushed in water.

In yet another embodiment, a method for using a menstrual flow absorbent disposable device to absorb excess menstrual fluid and to prevent leaks is described. The method includes the steps of providing the device, the device has a V-shape conforming to the space between the buttocks or intergluteal cleft of a human and is made of a flushable and flexible material. The method further includes inserting the pointed edge of the V-shaped device between the buttocks or gluteus maximus muscles of a user, placing a sanitary pad for absorbing the menstrual fluid, wherein the device is positioned near one end of the sanitary pad, and removing the device by pulling the device out from between the buttocks or gluteus maximus muscles, preferably while sitting on a toilet seat, and subsequently flushing the device.

In yet another embodiment, the device is constructed to be breathable and thin enough to offer discretion and prevent skin irritation.

In another aspect, the device maintains the V-shape during use without the need for intentional unfolding.

In yet another aspect, the device can absorb up to five times the weight thereof in fluid, demonstrating a high absorbency per unit weight.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of the menstrual flow absorbent disposable device of the present invention in a flat shape in accordance with the disclosed structure;

FIG. 2 illustrates the menstrual flow absorbent disposable device of the present invention in a V-shape in accordance with the disclosed structure;

FIG. 3 illustrates a perspective view showing preferred position and size of the menstrual fluid absorbent disposable device of the present invention relative to a sanitary pad in accordance with the disclosed structure;

FIG. 4 illustrates a perspective view showing placement of the menstrual flow absorbent disposable device of the present invention between the buttocks or gluteus maximus muscles along with a sanitary pad for absorbing menstrual fluid in accordance with one embodiment of the present invention; and

FIG. 5 illustrates an exemplary packaging of the sleep free device of the present invention in accordance with one embodiment of the present invention.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for an improved mensuration tool for absorbing excess body fluids to prevent leaks. Additionally, there is a long felt need in the art for an improved mensuration tool that can be used in place of or along with conventional mensuration tools and products such as sanitary pads and tampons. Moreover, there is a long felt need in the art for a menstrual device that can be used by women during all activities to absorb menstrual flow. Further, there is a long felt need in the art for a menstrual device that is V-shaped and made of absorbent material to absorb any leaking menstrual flow. Furthermore, there is a long felt need in the art for a menstrual device that fits between the buttocks or gluteus maximus muscles of a female to catch leaking menstrual flow. Also, there is a long felt need in the art for a novel menstrual tool that provides absorption for extended time periods. Finally, there is a long felt need in the art for a menstruation tool that is flexible, can be bent into V-shape and fits between the vagina and buttocks or gluteus maximus muscles to absorb menstrual flow.

The present invention, in one exemplary embodiment, is a method for using a menstrual flow absorbent disposable device to absorb excess menstrual fluid and to prevent leaks. The method includes the steps of providing the device, the device has a V-shape conforming to the space between the buttocks or gluteus maximus muscles of a human and is made of a flushable and flexible material. The method further includes inserting the pointed edge of the V-shaped device between the buttocks or gluteus maximus muscles of a user, placing a sanitary pad for absorbing the menstrual fluid, wherein the device is positioned near one end of the sanitary pad, and removing the device by pulling the device out from between the buttocks or gluteus maximus muscles for flushing.

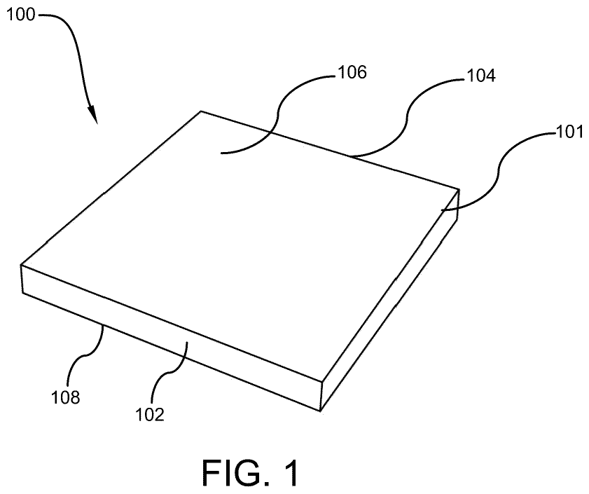

Referring initially to the drawings, FIG. 1 illustrates a perspective view of the menstrual flow absorbent disposable device of the present invention in a flat or unfolded shape in accordance with the disclosed structure. The menstrual flow absorbent disposable device or sheet 100 of the present invention is designed as a flushable and skin friendly device made of fabric or any other similar material and is adapted to be fixed between the buttocks or gluteus maximus muscles of a user to absorb excessive menstrual flow. More specifically, the menstrual fluid absorbent device or sheet 100 has generally square or rectilinear main body 101 and has a size of from about 2.5″ (L)×2.5″ (W) to about 3″ (L)×3.5″ (W) and is adapted to be bent in a V-shape for an easy fit between buttocks or gluteus maximus muscles (i.e., into the intergluteal cleft) of a user and along with use of sanitary pads.

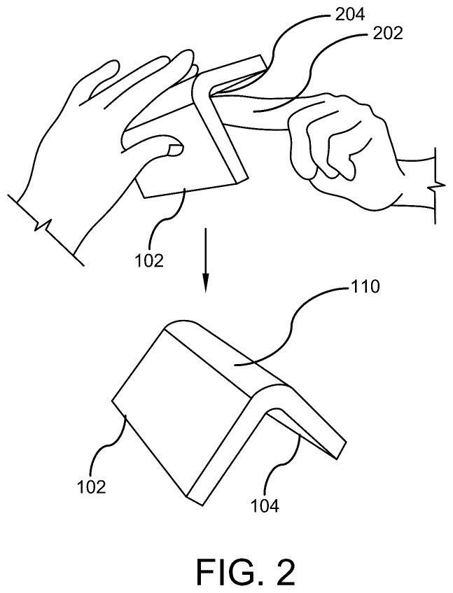

Referring now to FIGS. 1 and 2, the menstrual fluid absorption device or sheet 100 is bent or partially folded into a V-shape (Arrow B) for use to fit between the buttocks or gluteus maximus muscles. The bending or folding can be easily done by placing one or more fingers 202 at the center 204 of the device or sheet 100 and the side edges 102, 104 are folded towards each other to give a V-shape to the device or sheet 100. The V-shape of the absorbent device or sheet 100 helps in conforming to the contours of buttocks, or intergluteal cleft, of users, ensuring that the device or sheet 100 stays in place during various activities, including physical exercise and sleep.

Preferably, the menstrual fluid absorbent device or sheet 100 is made of superabsorbent biodegradable polymers, flushable cellulose fibers, and flushable nonwoven fabrics. The device or sheet 100 can easily disintegrate in water and are environmentally friendly. Further, the device or sheet 100 is breathable to prevent irritation and thin enough for discretion. Once device or sheet 100 has the V-shape, the V-shape is maintained without intentional unfolding of the device or sheet 100 by pulling the edges 102, 104 apart. In some embodiments, the device or sheet 100 is preconfigured and packaged in the V-shape and is not required to be folded to a V-shape by a user.

The top surface 106 and the bottom surface 108 of the device or sheet 100 are of same size and shape. Further, the device or sheet 100 can have a single layer or multiple layers to absorb excessive menstrual fluid of users of the device or sheet 100. The device or sheet 100 can absorb up to five times their weight in fluid and has a very high absorbency per unit weight.

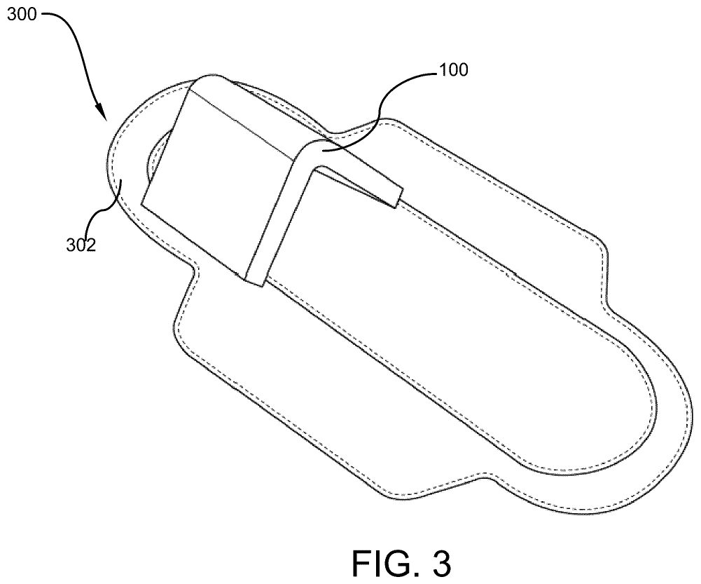

FIG. 3 illustrates a perspective view showing preferred position and size of the menstrual fluid absorbent disposable device of the present invention relative to a sanitary pad in accordance with the disclosed structure. The menstrual fluid absorbent disposable device or sheet 100 is placed between the buttocks or gluteus maximus muscles of a user and accordingly, is positioned near one end (i.e., proximal to a rearward end) 302 of the sanitary pad 300. The end 302 is positioned near the buttock of a user. The compact size and placement of the device or sheet 100 between the buttocks and the vagina, do not affect and impact the placement, stability, and efficacy of an adjacent or associated sanitary pad 300.

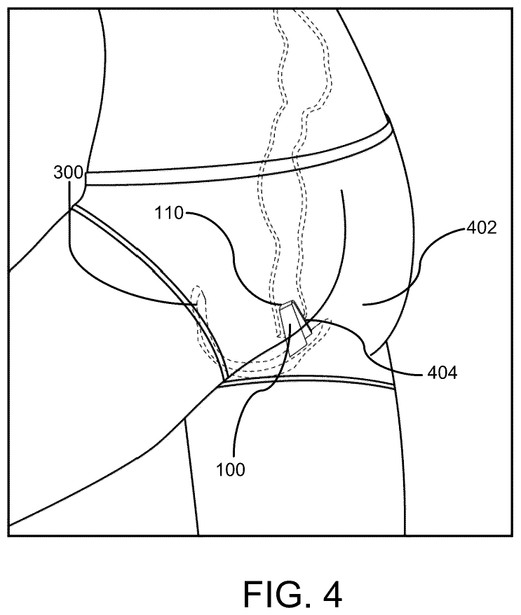

FIG. 4 illustrates a perspective view showing placement of the menstrual flow absorbent disposable device of the present invention between the buttocks or gluteus maximus muscles along with a sanitary pad for absorbing menstrual fluid in accordance with one embodiment of the present invention. For using the device or sheet 100, the device or sheet 100 is bent into V-shape as illustrated in FIG. 2. And the common pointed edge 110 is inserted in the space or intergluteal cleft 404 between the buttocks or gluteus maximus muscles 402 of the user. The device or sheet 100 remains stable to absorb any leaking menstrual fluid especially while the user using the device or sheet 100 is lying on their back. The device or sheet 100 is placed inside the sanitary pad 300 and therefore, functions as an additional barrier between bodily fluids and clothing/bedding to help preventing staining of clothing/bedding.

After use or during change of the sanitary pad 300, the absorbent device or sheet 100 can be pulled out from the buttocks or intergluteal cleft 404. Preferably, the user while sitting on a toilet seat can push and the device or sheet 100 can be easily pulled out and can be flushed. The menstrual flow absorbent device or sheet 100 is easy to use, remove, and provides an effective additional barrier between the menstrual fluid and the clothing of the user.

The V-shape fits comfortably and can be easily taken out or removed with minimal effort. The removal process is easy and painless, without requiring excessive force or causing discomfort. Further, the absorbent device or sheet 100 can be flushed and therefore is easy to dispose after use.

The sleep free device or sheet 100 of the present invention combines ergonomic design with specialized materials to provide a discreet, comfortable, and effective menstrual hygiene solution. The device or sheet 100 allows the users to do various activities, including sleep, without the worry of leaks, while also being flushable for convenient disposal.

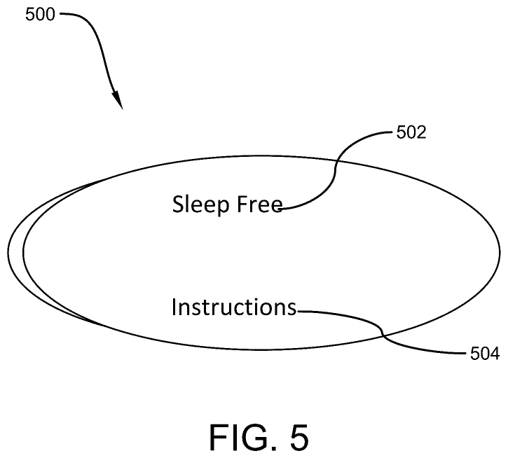

FIG. 5 illustrates an exemplary packaging of the sleep free device of the present invention in accordance with one embodiment of the present invention. The packaging 500 can be in the form of sachets or packets containing one sleep free device or sheet 100 or alternatively, can include a plurality of devices. Further, the packaging 500 can have a logo or indicia 502 disposed thereon along with instructions 504 to use the device or sheet 100.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “menstrual flow absorbent disposable device”, “menstrual fluid absorption device”, “sleep free device”, “absorbent device”, and “device” are interchangeable and refer to the menstrual fluid absorbent flushable device or sheet 100 of the present invention.

Notwithstanding the forgoing, the menstrual fluid absorbent flushable device or sheet 100 of the present invention can be of any suitable size and configuration as is known in the art without affecting the overall concept of the invention, provided that it accomplishes the above stated objectives. One of ordinary skill in the art will appreciate that the menstrual fluid absorbent flushable device or sheet 100 as shown in the FIGS. is for illustrative purposes only, and that many other sizes and shapes of the menstrual fluid absorbent flushable device or sheet 100 are well within the scope of the present disclosure. Although the dimensions of the menstrual fluid absorbent flushable device or sheet 100 are important design parameters for user convenience, the menstrual fluid absorbent flushable device or sheet 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A menstrual flow absorbent disposable device comprising:
an initially unfolded flexible fabric sheet having a top surface and a bottom surface;
wherein said flexible fabric sheet having an absorbent material;
wherein said flexible fabric sheet is flushable in a toilet;
wherein said flexible fabric sheet subsequently folded for placing a folded edge between the gluteus maximus muscles of a user;
wherein said flexible fabric sheet is held between a user's gluteus maximus muscles and within the user's intergluteal cleft for absorbing menstrual flow; and
further wherein said flexible fabric sheet is used in conjunction with a sanitary pad and is positioned between the sanitary pad and the user's intergluteal cleft.

2. The menstrual flow absorbent disposable device of claim 1, wherein said flexible fabric sheet is partially folded into a V-shape.

3. The menstrual flow absorbent disposable device of claim 2, wherein said partially folded said flexible fabric sheet is folded on a center line of said flexible fabric sheet.

4. The menstrual flow absorbent disposable device of claim 3, wherein said flexible fabric sheet having a material selected from the group consisting of biodegradable polymers, flushable cellulose fibers, and flushable nonwoven fabrics.

5. The menstrual flow absorbent disposable device of claim 4, wherein said flexible fabric sheet having a length from 2.5″ to 3.0″ and a width from 2.5″ to 3.5″.

6. The menstrual flow absorbent disposable device of claim 5, wherein said top surface and said bottom surface having the same size and shape.

7. The menstrual flow absorbent disposable device of claim 5, wherein said flexible fabric sheet disintegrates in water.

8. A menstrual flow absorbent disposable device comprising:
an initially unfolded fabric sheet having a top surface and a bottom surface;
wherein said flexible fabric sheet having an absorbent material;
wherein said flexible fabric sheet subsequently folded for placing a folded edge between the gluteus maximus muscles of a user;
wherein said flexible fabric sheet is held between a user's gluteus maximus muscles and within the user's intergluteal cleft for absorbing menstrual flow;
wherein said flexible fabric sheet is used in conjunction with a sanitary pad and is positioned between the sanitary pad and the user's intergluteal cleft; and
further wherein said flexible fabric sheet is positioned proximal to a rearward end of the sanitary pad.

9. The menstrual flow absorbent disposable device of claim 8, wherein said flexible fabric sheet is flushable in a toilet.

10. The menstrual flow absorbent disposable device of claim 8, wherein said flexible fabric sheet is partially folded into a V-shape.

11. The menstrual flow absorbent disposable device of claim 10, wherein said partially folded said flexible fabric sheet is folded on a center line of said flexible fabric sheet.

12. The menstrual flow absorbent disposable device of claim 8, wherein said flexible fabric sheet having a material selected from the group consisting of biodegradable polymers, flushable cellulose fibers, and flushable nonwoven fabrics.

13. The menstrual flow absorbent disposable device of claim 8, wherein said flexible fabric sheet having a length from 2.5″ to 3.0″ and a width from 2.5″ to 3.5″.

14. The menstrual flow absorbent disposable device of claim 8, wherein said top surface and said bottom surface having the same size and shape.

15. The menstrual flow absorbent disposable device of claim 9, wherein said flexible fabric sheet disintegrates in water.

16. A method of absorbing a menstrual flow, the method comprising the steps of:
providing an initially unfolded fabric sheet having a top surface and a bottom surface, wherein said flexible fabric sheet having an absorbent material;
folding said flexible fabric sheet into a V-shape along a folded edge;
placing said folded edge between the gluteus maximus muscles of a user, wherein said flexible fabric sheet is held between a user's gluteus maximus muscles and within the user's intergluteal cleft for absorbing menstrual flow;
positioning said flexible fabric sheet between a sanitary pad and the user's intergluteal cleft; and
further positioning said flexible fabric sheet proximal to a rearward end of the sanitary pad.

17. The method of absorbing a menstrual flow of claim 16, wherein said flexible fabric sheet is flushable in a toilet.

18. The method of absorbing a menstrual flow of claim 16, wherein said folded said flexible fabric sheet is folded on a center line of said flexible fabric sheet.

19. The method of absorbing a menstrual flow of claim 16, wherein said flexible fabric sheet having a material selected from the group consisting of biodegradable polymers, flushable cellulose fibers, and flushable nonwoven fabrics.

20. The method of absorbing a menstrual flow of claim 16, wherein said flexible fabric sheet having a length from 2.5″ to 3.0″ and a width from 2.5″ to 3.5″, and further wherein said top surface and said bottom surface having the same size and shape.
